(19) United States
(12) Patent Application Publication
(10) Pub. No.: US 2009/0287293 A1
(43) Pub. Date: Nov. 19, 2009

(54) MAGNETICALLY INDUCED RADIAL EXPANSION VASCULAR STENT

(76) Inventor: Robert Mailhot, JR., Trois-Rivieres (CA)

Correspondence Address: FRASER CLEMENS MARTIN & MILLER LLC, 28366 KENSINGTON LANE, PERRYSBURG, OH, 43551, US

(21) Appl. No.: 12/121,202

(22) Filed: May 15, 2008

(51) Int. Cl. A61F 2/82

(52) U.S. Cl. 623/1.15

(57) ABSTRACT

A magnetically induced radially expandable vascular stent for use inside a human body to hold open a stenosed vascular lumen. The stent comprises a flexible yet non elastic tubular main body, defining a peripheral wall having a radially outwardly expanded limit condition. A plurality of magnets are mounted in closely spaced wall pockets made in the main body peripheral wall. The relative orientation and position of the magnets are such that an equilibrium state is achieved corresponding to the tubular main body radially outward expanded condition, whereby the net effect of magnetic repulsion between the array of magnets is transformed into a synchronous mechanical radial expansion force of the stent tubular main body to an expanded stable condition thereof.

### FIELD OF THE INVENTION

This invention relates to implantable medical devices, such as stents placed in a human body after percutaneous balloon angioplasty, to hold open a stenosed vascular lumen and to maintain potency thereof In particular, this invention relates to systems for using magnetic components to stabilize the expanded diameter of stents in their in situ stenosed blood vessel position, while maintaining full performance thereof even after years of use.

### BACKGROUND OF THE INVENTION

A stent for transluminal implantation generally comprises metallic supports which are inserted into a part of the human body, for example the digestive tube but more notably by percutaneous route inside a blood vessel, usually the arteries in which case they are termed vascular stents. A stent is generally a cylindroid tube and is constructed and arranged to expand radially outwardly once in position within the body. It is usually inserted following percutaneous balloon angioplasty while it has a first relatively small diameter and implanted in a desired area, for example inside a stenosed peripheral or coronary artery section, then the stent is radially expanded in situ until it reaches a second diameter larger than the first diameter.

A balloon associated with a catheter can be used to provide the necessary interior radial force to radially expand the stenosed vessel so as to enable the stent to fit therein. Or alternately, self-expanding stents are also known which can expand from a first diameter to a radially larger second diameter without the use of a means for applying an interior radial force to them, for example a shape memory stent which expands to an implanted configuration upon being triggered by a temperature change.

Among the reasons why current stents are unreliable, there is the way the stents are constructed. Stents commonly have some form of structural ring elements. These are the portions of the stent that both expand and provide the radial strength. These ring elements are joined by links of various sorts. This combination of rings and links creates enclosed cells, and taken together, they create continuous loops. These loops can run around the circumference of the stent, or they can run in portions of the stent wall. Any modern stent pattern will show a variety of hoops, rings, loops, or cells, which can lose their elasticity and can break if subjected to some degree of compression or of crushing force. The stent thus loses its radial expansion torque, and there follows closure of the inner lumen thereof. This phenomenon is well known by vascular surgeons and is called “stent fracture”. Stent fracture is a serious problem, since ischaemy of the limb may then follow as a consequence, with possible further loss of the limb and even death.

It is known by medical practitioners that several blood vessels of the body, for example those close to the joints, are subjected to large bending biases generated during flexion of this joint (for example, the common femoral artery or the popliteal artery), thus causing a considerable crushing force at the level of the lumen of this vessel. It is also possible that an external compression force be applied at the level of a vessel close to two bony structures, during cyclical movements (for example, between sub-clavian vein and a first rib). Stent fracture hazard prevents or substantially limits operation of conventional stents in such conditions, in view of the serious repercussions that a stent fracture may generate.

The use of magnets to promote healing and reduce pain has been suggested in the prior art, i.e. where the magnetic field allegedly assists in improving post operative healing, or allegedly assists in alleviating pain due to muscle strain, tennis elbows, sore muscles, and the like. Stents with magnetic properties to allegedly encourage healing of a potentially damaged or weakened vessel, are also known.

Magnetism is one of the phenomena by which materials—such as nickel, iron, cobalt,—exert attractive or repulsive forces on other materials. Every electron, by its nature, is a small magnet; however, in a bar magnet, the electrons are aligned in the same direction, so they act cooperatively, creating a net magnetic field. A dipole is a common source of magnetic field, with a “south pole” and a “north pole”. Since opposite ends of magnets are attracted, the north pole of a magnet is attracted to the south pole of another magnet. A magnetic field contains energy, and physical systems stabilize into the configuration with the lowest level of energy. Therefore, when placed in a magnetic field, a magnetic dipole tends to align itself in opposed polarity to that field, thereby cancelling the net field strength as much as possible and lowering the energy stored in that field to a minimum. Hence, two identical bar magnets placed side-to-side normally lie North to South, resulting in a much smaller net magnetic field, and will resist any attempt to reorient them to point in the same direction. That is to say, a magnetic dipole in a magnetic field experiences a torque and a force which can be expressed in terms of the field and the strength of the dipole, i.e. its magnetic dipole moment.

Magnetic dipole moment quantifies the contribution of the system's internal magnetism to the external dipolar magnetic field produced by the system, i.e. the component of the external magnetic field that drops off close to one pole of a magnet with distance as the inverse square. Any dipolar magnetic field pattern is symmetric with respect to rotations around a particular axis, therefore the magnetic dipole moment that creates such a field is a vector with a direction along that axis. Any system possessing a net magnetic dipole moment will produce a dipolar magnetic field in the space surrounding the system. Magnetic moment can be visualized as a bar magnet which has magnetic poles of equal magnitude, but opposite polarity. Each pole is the source of magnetic force which weakens with distance. Since magnetic poles always come in pairs, their forces partially cancel each other because while one pole pulls, the other repels. This cancellation is greatest when the poles are close to each other, i.e when the bar magnet is short. The magnetic force produced by a bar magnet, at a given point in space, therefore depends on two factors: on both the strength of its poles, and on the distance separating them.

It is further known to provide an in vivo method for improving cardiac diastolic function of the left ventricle of the heart, comprising the steps of (a) operatively connecting magnetic components in a rest condition to the left ventricle of the heart, wherein these magnetic components feature physicochemical property and behavior for potentially exerting a radially outward expansive force or pressure to at least one part of wall region of the left ventricle during ventricular diastole; (b) allowing the heart to undergo ventricular systole, during which the potential radially outward expansive force or pressure of the magnetic components dynamically increases to a predetermined magnitude, and (c) allowing the heart to undergo ventricular diastole, during which the pre-determined magnitude of the potential radially outward expansive force or pressure of the magnetic components is dynamically converted into a corresponding kinetic radially outward expansive force or pressure applied to the wall region of the left ventricle, for reducing intracardiac hydrostatic pressure during the ventricular diastole, thereby improving the diastolic function of the left ventricle of the heart. However, it is noted that in such a method, there is a cyclical transfer of energy from the systolic stage to the diastolic stage of the overall cardiac cycle, which requires the assembly of magnetic components to demonstrate elasticity, so as to be able to dynamically change shape in a periodic cyclical fashion, in line with cyclical changes of shape of the heart during each heart beat.

### SUMMARY OF THE INVENTION

This invention relates to a magnetically induced radially expandable vascular stent for use inside a human body to hold open a stenosed vascular lumen, said stent comprising: a) a flexible, non elastic, tubular main body, defining a peripheral wall with an intake opening at one end thereof and a outlet opening at another end thereof opposite said one end and a free flow channel therebetween, said peripheral wall having a radially outwardly expanded limit condition; and b) magnetic means, continuously biasing said tubular main body toward said radially outwardly expanded limit condition in a stable equilibrium state.

Preferably, said magnetic means includes: a number of pocket members, integrally mounted into said main body peripheral wall in closely spaced apart fashion; and a corresponding number of magnet members, each magnet member sized and shaped to fit snugly into and trapped inside a corresponding one of said pocket members, so that a peripherally disposed array system of magnet members spaced from one another is formed; wherein said pocket members are oriented in such a fashion and are in sufficient number with associated said magnet members that each pair of said magnet members inside a corresponding closely spaced pair of said pocket members generate a repulsive force therebetween, whereby said array system of magnet members generates a stable equilibrium state of said flexible non elastic tubular main body corresponding to said expanded limit condition thereof.

Stent design according to the invention is such that axial polarization magnets are used, i.e. magnets with polarization being orthogonal to the stent radius, and thus parallel to the long axis of the blood vessel. This specific layout of magnets (which are preferably cylindroid) enables procurement of a compressed diameter as small as possible, an important consideration to promote percutaneous stent engagement before radial expansion thereof. Moreover, it is noted that a small angular deviation (small acute angle) between the long axis of the cylindroid magnets and the long axis of the registering blood vessel, would still make the present invention operative, i.e. the magnetic field interaction from the multiple magnets layout would still enable radial expansion of the present stent tubular body. These magnets are arranged in rows, each row made up of a minimum of three (3) magnets, which are placed equidistant to one another along a circumference of this row. All adjacent poles of magnets from a same row are of the same type, for example, “+” side closely spaced to a “+” side.

The next row consists of the same number of axial polarization magnets as the preceding one, being equidistant relative to one another along this row circumference. All adjacent poles of magnets of this row are also of the same type, for example, “−” side closely spaced to a “−” side. On the other hand, the orientation of these magnets polarity is the opposite to that of the preceding row, wherein all poles spacedly facing each other are of the same type. This specific configuration of polarity between magnets from adjacent rows facing spacedly from each other enables avoidance of self collapse of the stent. Moreover, the position of each magnet of this row at the level of the circumference is such that each magnet of the second row is preferably exactly in between those of the preceding row. This latter configuration in turn provides an optimal distribution of the support on all the circumference of the blood vessel wall where the stent is applied.

The stent thus consists of an array of rows of magnets, multiplying the number of rows according to the stent length that is required, and by applying the hereinabove principles.

Said tubular wall may be double-layered, defining a radially outward first layer and a radially inward second layer, both said first and second layers being attached to one another except at discrete areas corresponding to said pocket members. This tubular wall could then be made from a material selected from the group comprising polyester and polytetrafluoroethylene (or ePTFE).

Alternately, said tubular wall is a tubular lattice. In such a case, said tubular lattice could include a plurality of elongated arms closely spaced from one another in successive rows each of at least three arms, in an open honeycomb pattern, said arms being hollow, each of said arm hollow forming one of said pocket members for receiving a corresponding one of said magnet members, and further including hinge means, interconnecting each successive rows of said elongated arms to enable radial expansion of said tubular lattice

For example, there are between nine (9) and forty five (45) said pocket members, with a corresponding number of said magnet members, depending inter alia on the size and length of the tubular lattice.

The invention also relates to a magnetic system for radially expanding a flexible vascular tube for use as an in vivo stent to hold open a stenosed vascular lumen, the tube being flexible and non elastic and defining a peripheral wall with an intake opening at one end and an outlet opening at another end opposite said one end and a free flow channel therebetween, the main body having a variable diameter wherein the main body further defines a radially outwardly expanded limit condition; wherein said magnetic system consists of a number of pocket members, for integrally mounting into the stent tube main body peripheral wall; and a corresponding number of magnet members, each magnet member sized and shaped to fit snugly into and being trapped inside a corresponding one of said pocket members, so that a peripherally disposed array system of said magnet members spaced from one another is formed; wherein said pocket members are oriented in such a fashion and are in sufficient number with associated said magnet members that each row of said magnet members inside a corresponding closely spaced row of said pocket members generate a repulsive force therebetween, whereby said array system of magnet members for generating a stable equilibrium state of the flexible non elastic tube main body corresponding to the radially outwardly expanded limit condition thereof.

Preferably, said magnet member is a cylindroid magnet.

Accordingly, the stent of the present invention is made from a tubular flexible main body made from material which will not impede magnetic flows such as ePTFE or a synthetic material such as polyester, with a radially adjustable diameter being enabled by a plurality of small magnets integrally mounted in a corresponding number of complementarily sized pockets made in the peripheral wall of the stent main body. The relative orientation of the magnets pockets relative to one another, and the relative magnetic field generating orientation of each magnet in its pocket, are such that the resultant magnetic vector force produced by the magnets is one of a continuous radially outward expansion of the flexible tubular main body. The combined intensity of magnetic force generated by the plurality of magnets will be sufficient to apply radially outward bias against the stenosed blood vessel.

In accordance with a first embodiment of the invention, there is provided a two-layer tubular flexible stent sheath, defining a first radially outward layer and a second radially inward layer. The diameter of the sheath after magnetic expansion is slightly greater than the optimal functional diameter of the blood vessel section into which the stent is installed, to ensure friction fit stable positioning thereof and prevent accidental shifting with time along the blood vessel.

To install this two layer flexible sheath stent, there may be used a cylindroid catheter inserted non-invasively through a small puncture made in the skin and in the blood vessel distally from the stenosed blood vessel segment. The patient does not need to be asleep during this intervention, as local anesthetics are usually sufficient The grips of the catheter with metallic guide wire grabs the present stent at the leading end thereof and maintains the tubular body thereof in a radially inward inoperative condition, diametrally smaller than the lumen of the blood vessel, against the radially outward resultant bias of the magnets array. The stent at the leading end of the catheter is pushed along the blood vessel, until the stent comes in transverse register with the stenosed segment of this blood vessel. At this point, the catheter grips release their grip on the stent main body, thus enabling the stent magnets array to transform magnetic repulsion forces into a radially outwardly mechanical force onto the stent flexible main body, up to its radially outwardly expanded operative stable limit condition.

The two layers of the stent sheath may be attached to one another for example by glue, by stitching, or even fused to one another, when the stent is made, except for the discrete areas forming the magnet receiving pockets. The magnets will then be trapped between the unattached portions of two layers of the stent sheath in closely spaced fashion—think ravioli pasta, and their meat receiving pockets. Each sheath main body pocket is sized and shaped to complementarily receive one magnet, so that the magnet will remain stationary: in particular, accidental translational, yaw or tilt motion should be prevented, and also rotational motion in the case of bar magnets, so as to constantly maintain the same magnetic field orientation.

Each magnet may be preferably cylindroid. There may be for example between nine (9) and forty five (45) pockets, with corresponding number of magnets, depending inter alia on the size and shape of the stent sheath. Each magnet is placed in a selected stent body pocket in such a way that magnetic dipole moments are generated between rows of at least three magnets that are closely spaced from one another. Numerous repulsive force dipoles are generated, i.e. pairs of magnetic forces of approximately equal magnitude but of same polarity separated by a small distance, wherein each magnet from a pair of dipole magnets are submitted to repulsive forces that bias away from one another these at least three magnets from each row of dipole magnets. The relative position of these pockets relative to other pockets, and the relative orientation of these magnets in their pocket, generate an overall array of magnetically repulsing forces that transform into a resultant vector of mechanical force corresponding to a continuously acting radial expansion force applied onto the two interconnected layers of the flexible stent sheath, thus radially outwardly biasing the stent sheath. The type and number of these magnets are selected such as to have sufficient radially outward force to radially outwardly bias the blood vessel, thus frictionally interlocking the tubular stent body to the inner wall of the stenosed blood vessel section.

In accordance with an alternative embodiment of the invention, there is provided a single layer tubular lattice main body, such as those used in conventional stents. This tubular lattice is generally open, defining honeycomb like pattern. As in the first embodiment of stent, the expanded stable diameter of the lattice main body after magnetic radial expansion is slightly greater than the optimal diameter of the blood vessel segment into which the stent is to be mounted. Delivery and installation thereof can be the same as with the first embodiment of stent.

In this alternate embodiment of stent, the size of the magnet pockets is reduced to the smallest possible, as well as the size of the mesh providing structural integrity between each pair of closely spaced magnets, wherein the overall diameter thereof in its radially inward limit position is as compact as possible to facilitate this stent installation through the blood vessel lumen. Accordingly, the radially inward limit diameter of this stent should be substantially smaller than that of the first hereinabove embodiment. A major portion of the tubular lattice will therefore be modified to make it hollow. Inner pockets each having a size and shape complementary to a given cylindroid magnet, will be provided, such that each magnet will remain taut in place in its corresponding pocket while being prevented from accidental tilting, yaw or translational motion. The relative position of these magnets holding pockets, and the relative orientation of the magnets inside their respective pockets, will generate dipole moments between closely spaced pairs of magnets that will bring about a resultant vector of mechanical force consisting of a radially expanding biasing force on the overall flexible tubular lattice toward a stable expanded condition.

Each magnet may be for example a bar magnet, or preferably a cylindroid magnet, or other suitable shape; and preferably made of Neodymium-Iron-Boron alloy (NdFeB), ideal for the force of magnetic field generated relative to the magnets' weight.

### BRIEF DESCRIPTION OF THE DRAWINGS

FIG. 1a is a perspective view of a first embodiment of tubular lattice type vascular stent, in its unbiased retracted state;

FIG. 1b is a view similar to FIG. 1a, but with the stent shown in its radially expanded stable operative condition;

FIG. 1c is a schematic plan view of the stent of FIGS. 1a-1b, suggesting the magnetic interaction between the magnets integral to said stent;

FIG. 2a is a perspective view of a second embodiment of tubular vascular stent, in its unbiased retracted inoperative state;

FIG. 2b is a view similar to FIG. 2a, but with the stent shown in its radially expanded stable operative condition;

FIGS. 3 and 4 are cross-sectional views taken along lines 3-3 and 4-4 respectively of FIG. 2b;

FIGS. 5 and 6 are cross-sectional views taken along lines 5-5 and 6-6 respectively of FIG. 2a;

FIG. 7 is an enlarged cross-sectional view taken along lines 7-7 of FIG. 1a;

FIG. 8 is an enlarged cross-sectional view taken along lines 8-8 of FIG. 1b;

FIG. 9 is a perspective view of a prior art catheter tool used to deliver stents into a vascular segment of a sick person, in a non-invasive fashion;

FIG. 10 is an enlarged longitudinal sectional view of a section of blood vessel, the blood vessel disclosing a a radially inwardly projecting narrowed section, and the blood vessel further showing therein the head at the distal end portion of the catheter of FIG. 9 carrying the stent embodiment of FIGS. 1a-1b in the retracted inoperative condition thereof; and

FIG. 11 is a view similar to FIG. 10, but with the stent embodiment of FIG. 1b in its radially expanded stable condition providing radially outward bias against said blood vessel radially inwardly projecting narrowed (stenosed) section, and said tubular stent further shown being released from the catheter head.

### DETAILED DESCRIPTION OF THE EMBODIMENTS OF THE INVENTION

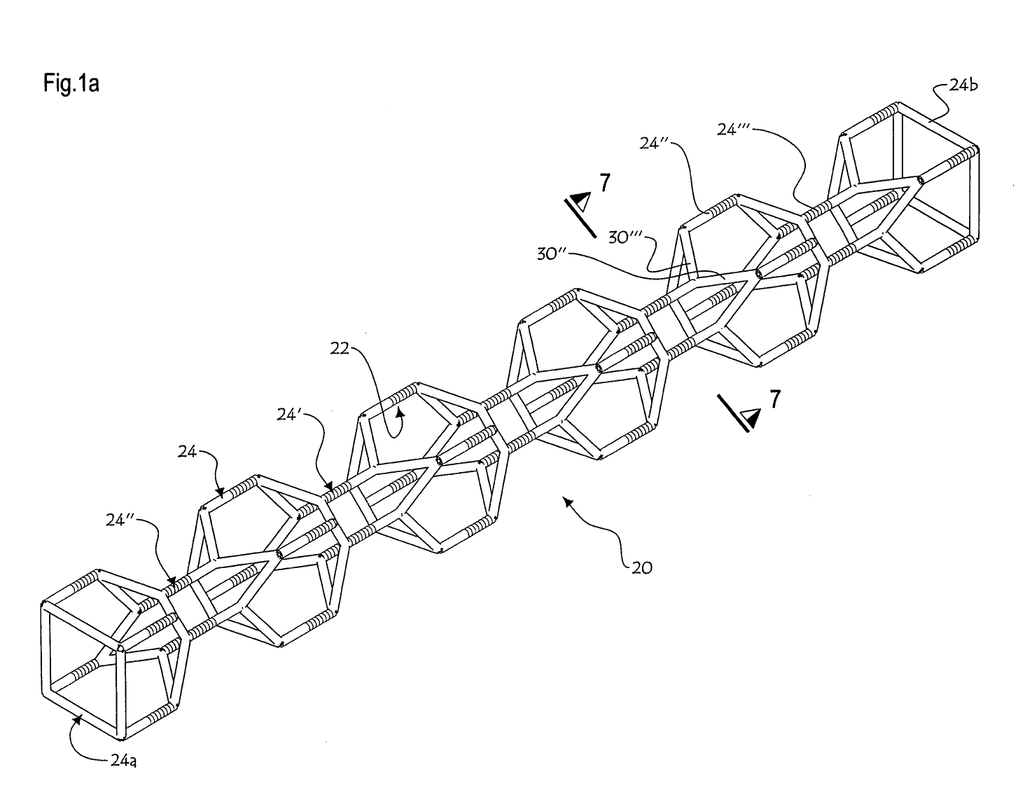

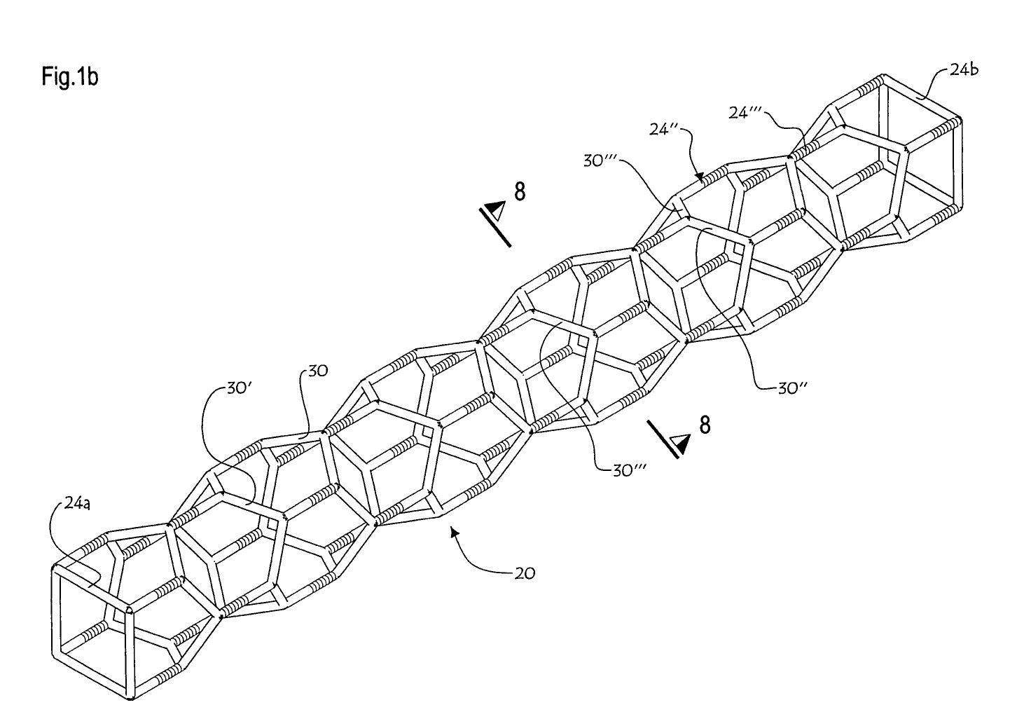

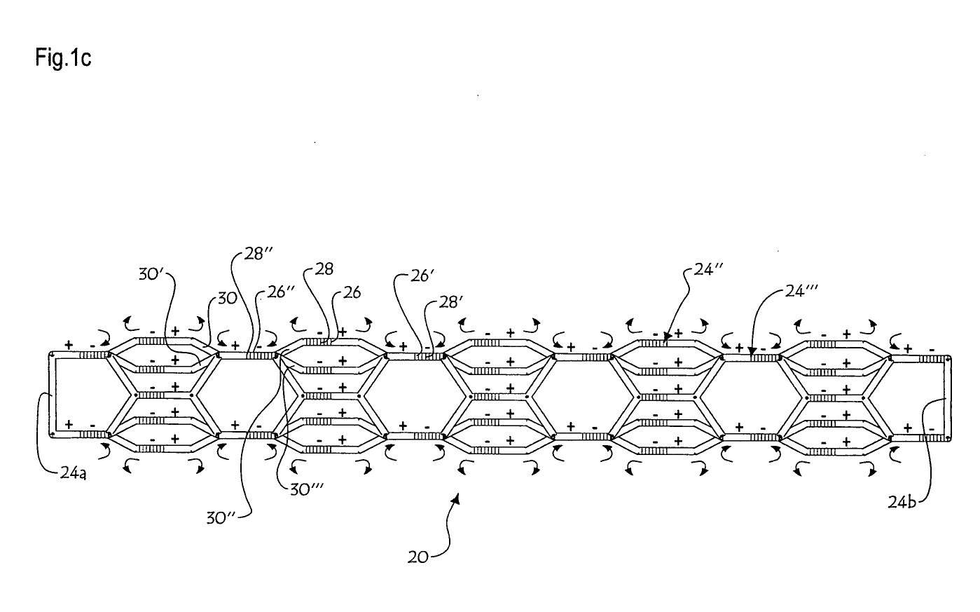

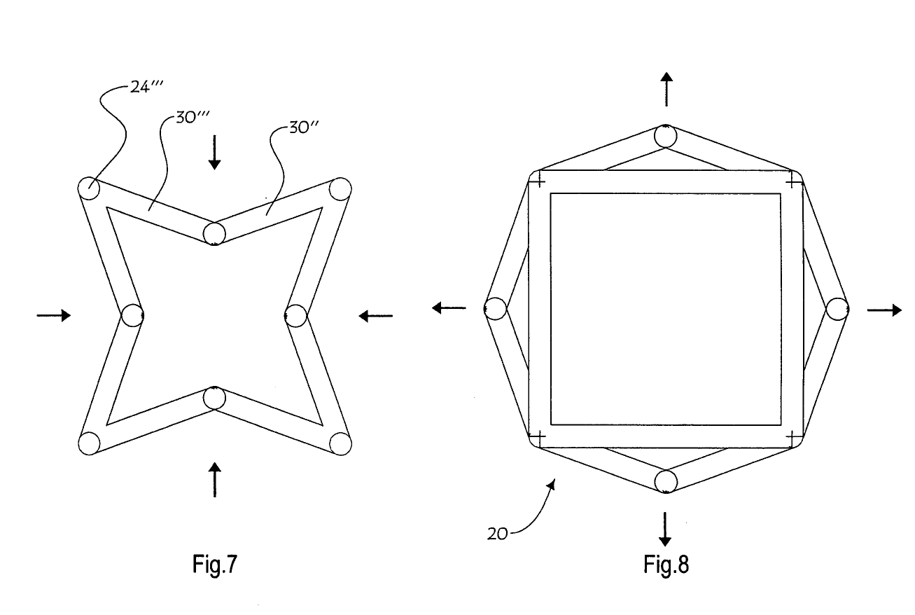

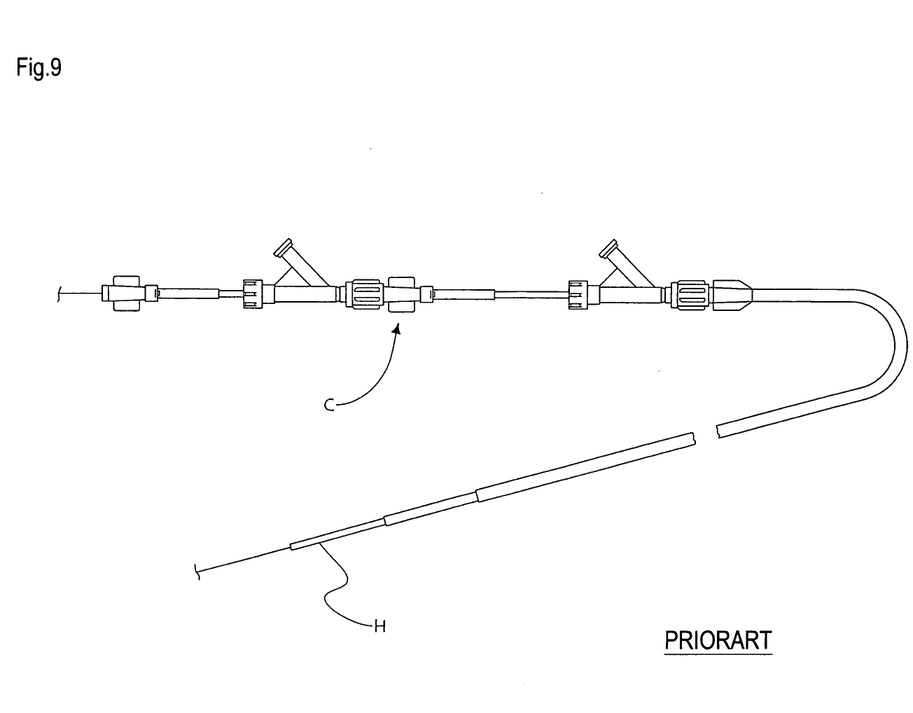

The tubular lattice like vascular stent 20 of FIGS. 1a-1b-1c and 7-8 consists of a honeycomb-like pattern tube 22. Tube 22 includes a plurality of elongated arms 24, 24′, 24″, 24′″, . . . each spacedly interconnected at their opposite ends to one or two other arms 24, 24′, 24″, 24′″, wherein a generally open structure is obtained. Each elongated arm 24, 24′, . . . may be for example cylindroid, as illustrated. The interconnecting points 30, 30′, 30″, 30′″, . . . between each pair of successive arms 24, 24′, . . . should be made from a flexible, yet non elastic material, for example a suitable synthetic material such as non elastic ePTFE, as suggested by the sequence of FIGS. 1a (retracted stent) and 1b (radially expanded stent).

Each arm 24, 24′, . . . comprises at least a portion thereof which is hollow, defining therein a pocket 26, 26′, 26″, . . . sized to snugly accommodate a correspondingly sized and shaped tubular dipole magnet member 28, 28′, 28″, 28′″, . . . (FIG. 1c). For example, there may be the number of rows each of at least three (3) magnets members 28, 28′, . . . in a given stent 20 with a corresponding number of tube pockets 26, 26′, although other suitable numbers are also envisioned, for example up to fifteen (15) rows of three magnets per each row. However, the polygonal (e.g. quadrangular as shown) arm structure 24A, 24B, at each of the two opposite ends of elongated tube 22, do not carry any magnet member as suggested in FIG. 1a. Magnet members 28, 28′ may be for example a bar magnet, but preferably a cylindroid magnet member.

In the inoperative, radially inward condition of the stent tube 22, the North end (+) of each magnet 28, . . . comes in closely spaced register with the North end (+) of each other magnet 28′, from a given successive row of magnets 28, 28′, 28″; . . . and similarly, the South end (−) of each magnet 28″, . . . comes in closely spaced register with the South end (−) of each magnet 28′″from a given successive row of magnets 28″, 28′″, . . . . In this way, a repulsive force is generated between each row of closely spaced successive magnets 28, 28′, 28″, and between each adjacent closely spaced pairs of magnets from a same row.

As these magnets 28, 28′, are trapped inside their corresponding pockets 26, 26′, arms 24, 24′, . . . become spread apart from one another due to the resultant vector force from the synchronized combined magnetically induced repulsive forces being transformed into mechanical expansion force applied at tubular interconnection points 30, 30′. The latter thus form hinge elements. The selected relative positioning of the arm pockets 26, 26′, in stent tube 28 is such that a radially outward expansion force is applied to the flexible non elastic tube 22. At that point, a stable magnetic dipole equilibrium condition is achieved at a predetermined operational expanded diameter of tube 22.

This operational expanded diameter of flexible tube 22 corresponds to a radially outwardly expanded condition of flexible tube 22, enabled by hinge elements 30, 30′, without any radially outward stretching of the material of tube 22. Tube 22 is of such construction and layout as to not being able to radially outward stretch under magnetically induced synchronous mechanical expansion forces, to provide optimal operational effectiveness of the stent 20 in the stenosed vascular segment.

Preferably, each magnet member 28, 28′ . . . is enclosed and sealed into a bead made preferably from titanium, silicone, polyurethane, or other suitable enclosing biohazard capsule, to shield biological material such as blood from accidental hazardous contamination, oxydation, or leak from the magnet member material to the blood or other biological part.

Stent tube 22 is sized to pass freely through a stenosed vascular segment, in a radially inward retracted inoperative condition of FIG. 1a, but to transversely radially engage with the inner wall of the stenosed segment in its radially outwardly expanded operative condition in friction fit fashion.

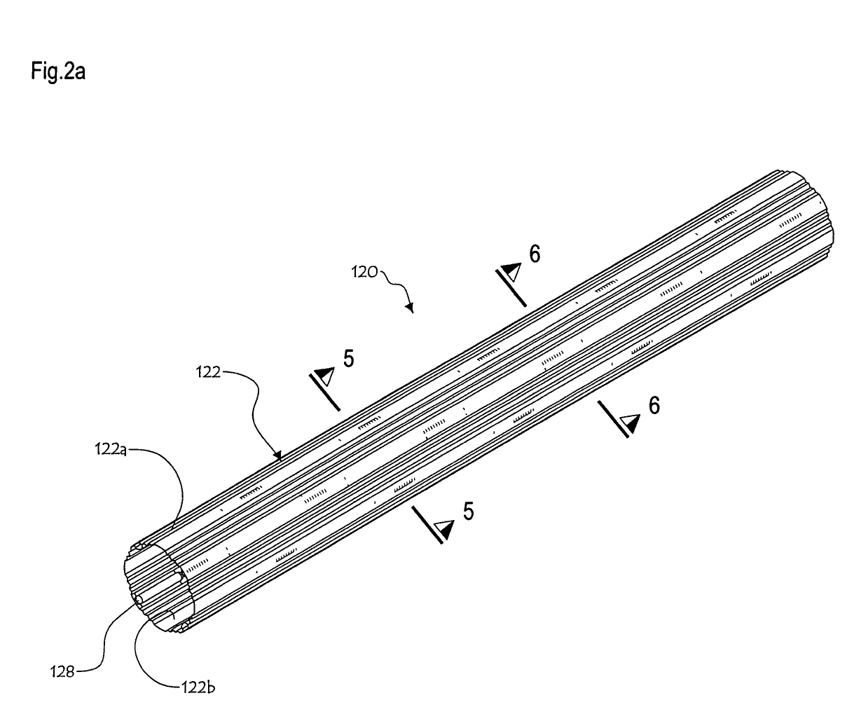

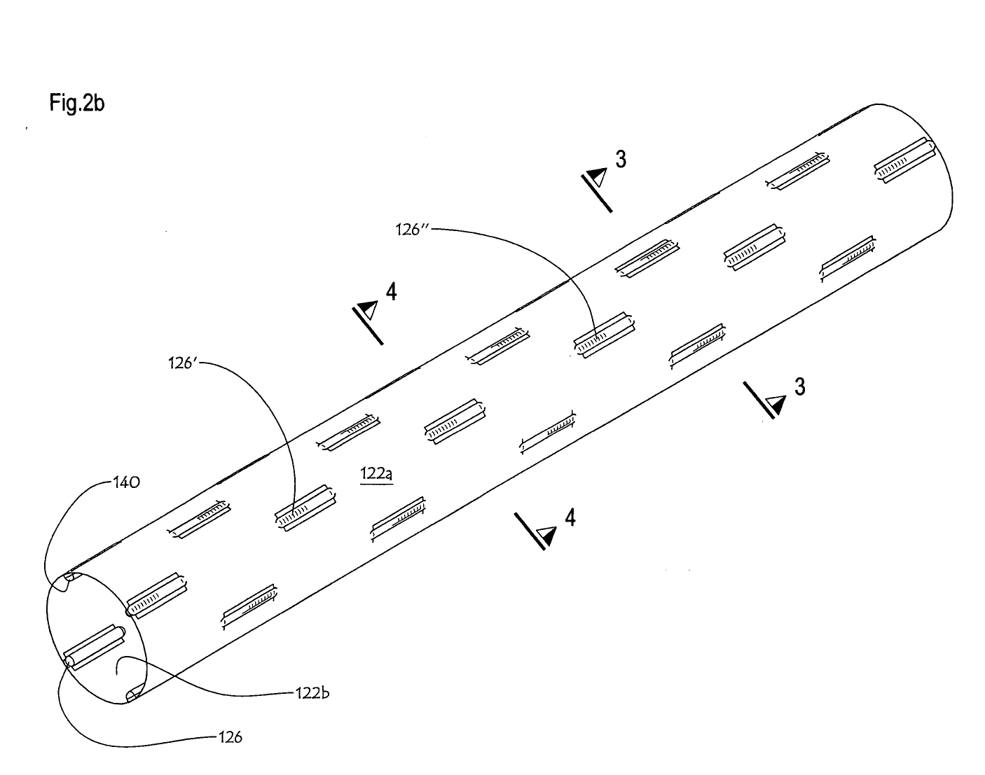

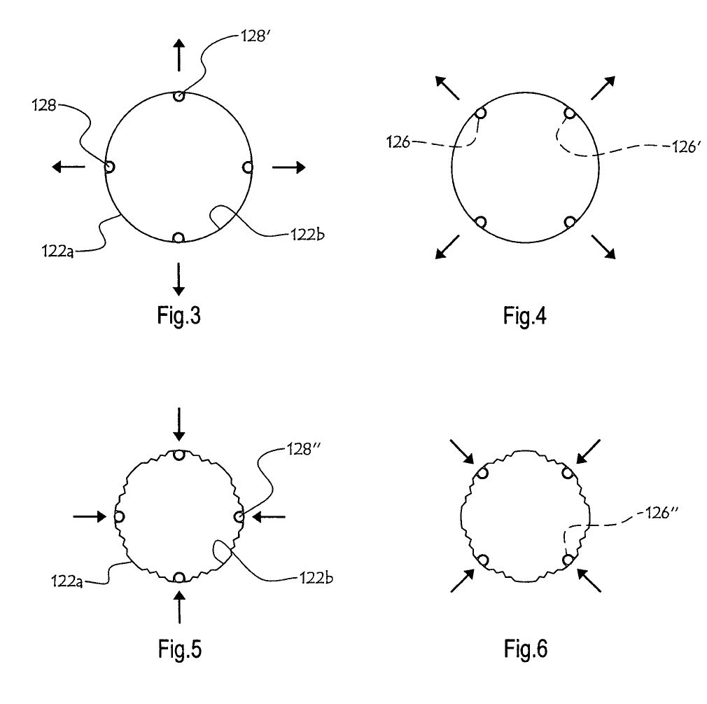

In the second embodiment of stent 120 shown in FIGS. 2a, 2b and 3 to 6, there is disclosed a substantially continuous cylindroid tube 122. Tube 122 is made from a flexible yet non elastic material, for example a flexible ePTFE material that cannot stretch radially outwardly. Preferably, tube 122 is double layered, defining a radially outward layer 122A, a radially inward layer 122B, and a gap 140 therebetween.

Gap 140 is closed along a substantial portion of tube 122, for example by stitching or glueing the two layers 122A, 122B to one another, except for a plurality of discrete pocket areas 126, 126′, 126″, . . . . As with the first embodiment of stent 20, each pocket 126, 126′, . . . is sized and shaped to snugly receive a corresponding magnet 28, 28′, . . . .

In both embodiments of stents 20, 120, magnets 28, 28′, . . . and 128, 128′, . . . are prevented from accidental tilt, translational or yaw movements relative to the tube 22, 122, respectively by being trapped inside their pockets 26, 26′ . . . and 126, 126′, . . . . Moreover, accidental rotational movement of a bar magnet (but not a cylindroid magnet) could compromise the performance of a stent fitted with bar magnets, so this rotational movement should be prevented with stents having bar magnets, for example by sufficient frictional interlock of each magnet with the material of the corresponding tube pocket.

FIGS. 2a and 5-6 show the flexible, non elastic tube membrane 122 being in its wrinkled, radially inward, inoperative condition, while FIGS. 2b and 3-4 show tube membrane 122 being in its radially outwardly expanded, yet unstretched, operative condition. Other operational features discussed with respect to the first embodiment of stent 20 are also applicable to the present stent 120.

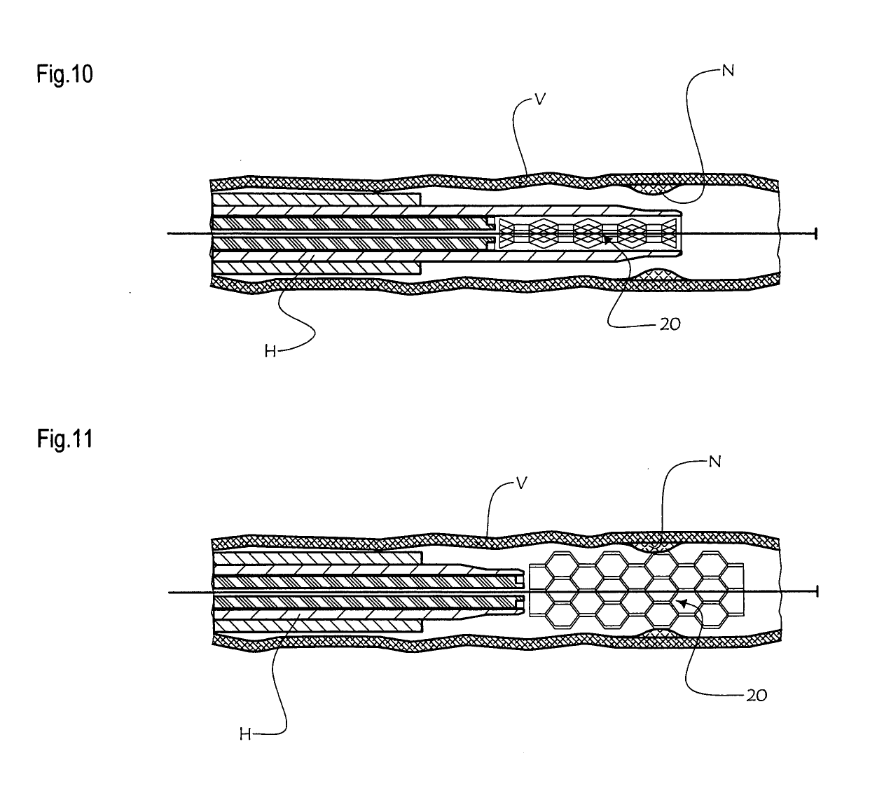

FIGS. 10 and 11 show in longitudinal section a blood vessel V with a portion thereof having a radially inward narrowing N. i.e. the stenosed part where plaques have become embedded to the inner wall of the blood vessel by which the lumen diameter of the blood vessel becomes narrower. We also see in FIG. 10 of the drawings that in the radially inward, inoperative condition of the represented first embodiment of stent 20, the diameter thereof is much less than the lumen diameter of the blood vessel stenosed part N, whereas in the radially expanded, operative condition thereof represented in FIG. 11, the stent 20 applies a radially outward force to the stenosed part N so as to become frictionally secured thereabout, FIG. 12 shows a conventional catheter guide wire delivery system for minimal invasive surgical delivery of the stent 20 or 120 to a vascular stenosed section of blood vessel as suggested in FIGS. 10-11.

I claim:

1. A magnetically induced radially expandable vascular stent for in vivo use to hold open a stenosed vascular lumen, said stent comprising:
a) a flexible, non elastic, tubular main body, defining a peripheral wall with an intake opening at one end thereof and a outlet opening at another end thereof opposite said one end and a free flow channel therebetween, said peripheral wall having a radially outwardly expanded limit condition; and
b) magnetic means, continuously biasing said tubular main body toward said radially outwardly expanded limit condition in a stable equilibrium state.

2. A vascular stent as in claim 1,
wherein said magnetic means includes:
a) a number of rows of pocket members, integrally mounted into said main body peripheral wall in closely spaced fashion; and
b) a corresponding number of rows of magnet members, each magnet member sized and shaped to fit snugly into and trapped inside a corresponding one of said pocket members, so that a peripherally disposed array system of magnet members spaced from one another is formed;
wherein said pocket members are oriented in such a fashion and are in sufficient number with associated said magnet members, that each row of said magnet members inside a corresponding closely spaced row of said pocket members generate a repulsive force therebetween, whereby said array system of magnet members generates a stable equilibrium state of said flexible non elastic tubular main body corresponding to said expanded limit condition thereof.

3. A vascular stent as in claim 2,
wherein said tubular wall is generally continuous and is double-layered, defining a radially outward first layer and a radially inward second layer, both said first and second layers being attached to one another except at discrete areas corresponding to said pocket members.

4. A vascular stent as in claim 3,
wherein said tubular wall is made from a material selected from the group comprising ePTFE and polyester.

5. A vascular stent as in claim 2,
wherein said tubular wall is a tubular lattice.

6. A vascular stent as in claim 5,
wherein said tubular lattice includes a plurality of elongated arms closely spaced from one another in successive rows each of at least three arms, in an open honeycomb pattern, said arms being hollow, each of said arm hollow forming one of said pocket members for receiving a corresponding one of said magnet members, and further including hinge means, interconnecting each successive pair of said elongated arms to enable radial expansion of said tubular lattice.

7. A vascular stent as in claim 2,
wherein there are between nine (9) and forty five (45) said pocket members, with a corresponding number of said magnet members.

8. A magnetic system for radially expanding a flexible vascular tube for use as an in vivo stent to hold open a stenosed vascular lumen, the tube being flexible and non elastic and defining a peripheral wall with an intake opening at one end and a outlet opening at another end opposite said one end and a free flow channel therebetween, the main body having a variable diameter wherein the main body further defines a radially outwardly expanded limit condition;
wherein said magnetic system consists of
a) a number of rows of pocket members, for integrally mounting into the stent tube main body peripheral wall; and
b) a corresponding number of rows of magnet members, each magnet member sized and shaped to fit snugly into and being trapped inside a corresponding one of said pocket members, so that a peripherally disposed array system of said magnet members spaced from one another is formed;
wherein said pocket members are oriented in such a fashion and are in sufficient number with associated said magnet members that each row of said magnet members inside a corresponding closely spaced row of said pocket members generate a repulsive force therebetween, whereby said array system of magnet members for generating a stable equilibrium state of the flexible non elastic tube main body corresponding to the radially outwardly expanded limit condition thereof.

9. A magnetic system for vascular stent as in claim 8,
wherein there are between nine (9) and forty five (45) said pocket members, with a corresponding number of said magnet members.

10. A magnetic system as in claim 8,
wherein each said magnet member is a cylindroid magnet member.

11. A magnetic system as in claim 2,
wherein each said magnet member is a cylindroid magnet member.

12. A vascular stent as in claim 2,
wherein there are between nine (9) and forty five (45) said pocket members, with a corresponding number of said magnet members.

13. A vascular stent as in claim 2,
wherein each said magnet member is enclosed and sealed into a biohazard capsule.

14. A magnetic system as defined in claim 8,
wherein each said magnet member is enclosed and sealed into a biohazard capsule.

15. A vascular stent as in claim 13,
wherein said biohazard capsule is a bead made from a material selected from the group comprising titanium, silicone, and polyurethane.

16. A magnetic system as defined in claim 14,
wherein said biohazard capsule is a bead made from a material selected from the group comprising titanium, silicone, and polyurethane.

17. A magnetic system as in claim 8,
wherein there are at least three said magnet members in each of said rows of magnet members.

18. A vascular stent as in claim 2,
wherein there are at least three said magnet members in each of said rows of magnet members.

19. A vascular stent as in claim 2,
wherein said magnet members are made of NdFeB alloy.

20. A magnetic system as in claim 8,
wherein said magnet members are made of NdFeB alloy.
